(19) United States
(12) Patent Application Publication
(10) Pub. No.: US 2024/0188423 A1
(43) Pub. Date: Jun. 6, 2024

(54) ORGANIC COMPOUNDS AND LIGHT EMITTING DEVICES

(71) Applicants: Beijing BOE Technology Development Co., Ltd., Beijing (CN); BOE Technology Group Co., Ltd., Beijing (CN)

(72) Inventor: Tieshi WANG, Beijing (CN)

(21) Appl. No.: 17/781,890

(22) PCT Filed: May 25, 2021
(86) PCT No.: PCT/CN2021/095870

§ 371 Date: Jun. 2, 2022

(51) Int. Cl. H10K 85/60; C07D 333/36; C09K 11/06; H10K 50/15; H10K 50/17

(52) U.S. Cl. CPC H10K 85/636; C07D 333/36; C09K 11/06; C09K 2211/1018; H10K 50/15; H10K 50/17; H10K 85/655

(57) ABSTRACT

The present disclosure provides an organic compound and a light emitting device, and relates to the field of display technologies. A structure of the organic compound is as shown in Formula 1, Formula 2 or Formula 3. The present disclosure can improve a hole transport rate of the light emitting device.

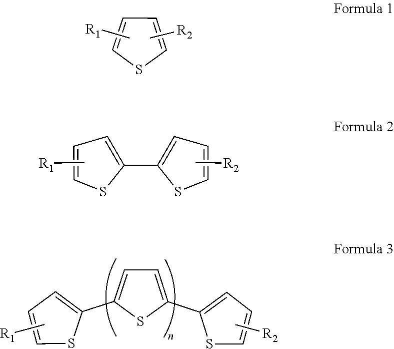

### TECHNICAL FIELD

The present disclosure relates to the field of display technologies, and in particular, to organic compounds and light emitting devices.

### BACKGROUND

Quantum dots, as a new type of luminescent materials, have advantages of high light color purity, high luminescent quantum efficiency, adjustable luminescent color, long service life, etc. Therefore, quantum dot light emitting devices with light emitting layers using quantum dot materials now have become a main direction of research on new display devices. However, since an electron transport rate of existing quantum dot light emitting devices is greater than a hole transport rate of the quantum dot light emitting devices, a carrier injection imbalance of the quantum dot light emitting devices is caused, which reduces device performance.

### SUMMARY

An object of the present disclosure is to provide organic compounds and light emitting devices, which can improve a hole transport rate of the light emitting devices.

According to an aspect of the present disclosure, there is provided an organic compound, where a structure of the organic compound is as shown in Formula 1, Formula 2 or Formula 3:

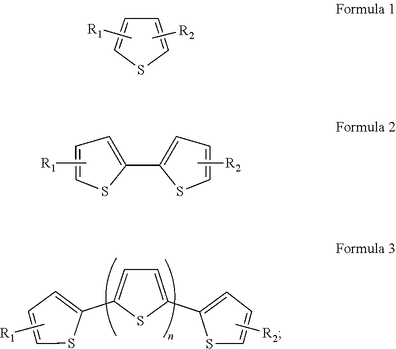

where n is an integer greater than or equal to 1;
each of R₁ and R₂, independently, is selected from hydrogen, a substituted or unsubstituted carbazolyl group, and a substituted or unsubstituted diphenylamino group, and R₁ and R₂ are not simultaneously hydrogen.

In some embodiments, the structure of the organic compound is as shown in Formula 1-1, Formula 2-1 or Formula 3-1:

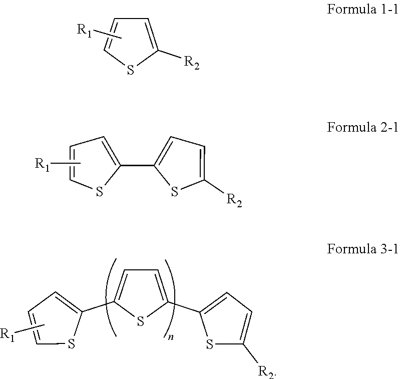

In some embodiments, the structure of the organic compound is as shown in Formula 1-2, Formula 2-2 or Formula 3-2:

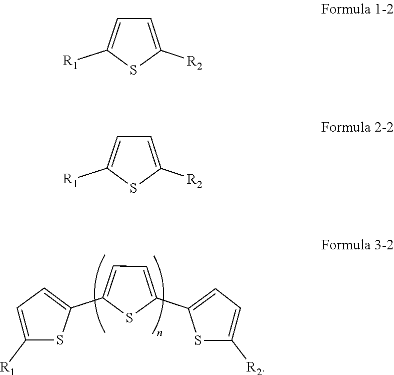

In some embodiments, each of R₁ and R₂, independently, is selected from hydrogen or following groups:

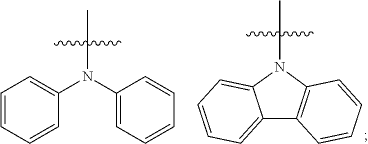

where

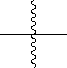

represents a chemical bond.

In some embodiments, n is an integer less than or equal to 10.

In some embodiments, the organic compound is selected from a group consisting of following structural formulas:

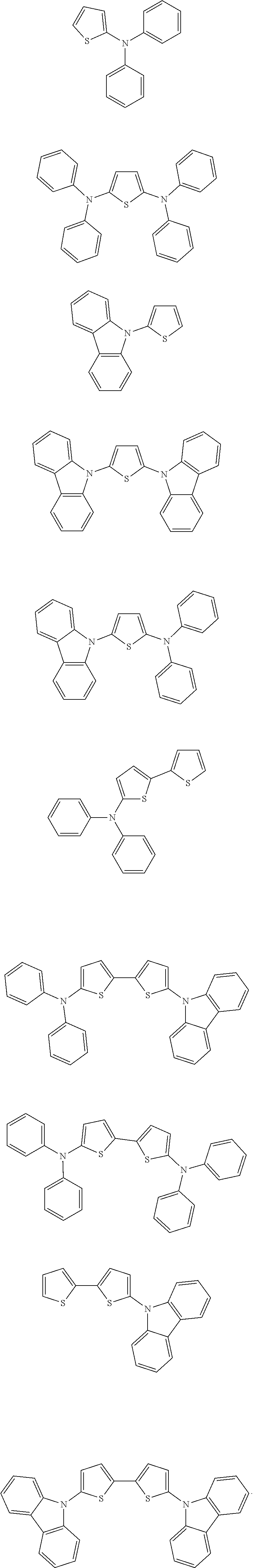

In some embodiments, R₁ and R₂ are the same as each other.

According to an aspect of the present disclosure, there is provided a light emitting device, including:
an anode and a cathode disposed opposite to each other;
a light emitting layer disposed between the anode and the cathode;
a hole function layer disposed between the anode and the light emitting layer and including the organic compound as described above.

In some embodiments, the hole function layer includes a hole transport layer, and the hole transport layer includes the organic compound.

In some embodiments, the hole function layer includes a hole injection layer, and the hole injection layer includes the organic compound.

In some embodiments, the light emitting device is a quantum dot light emitting device.

The organic compounds and the light emitting devices according to the present disclosure include thienyl groups, and substituents R₁ and R₂ bonded to the thienyl groups. The thienyl groups can impart a hole transport property to materials. R₁ and R₂ are each independently selected from hydrogen, a substituted or unsubstituted carbazolyl group, and a substituted or unsubstituted diphenylamine group, and R₁ and R₂ are not simultaneously hydrogen, thereby imparting a hole injection property to the materials, further making the materials have both the hole transport and injection properties, and improving a hole transport rate. The organic compound is used for preparing quantum dot light emitting devices, which can improve a carrier injection balance of the devices and increase performance of the devices.

### BRIEF DESCRIPTION OF THE DRAWINGS

FIG. 1 is a schematic diagram illustrating a light emitting device according to an embodiment of the present disclosure;

FIG. 2 is another schematic diagram illustrating a light emitting device according to an embodiment of the present disclosure.

Description of reference signs: 1. anode; 2. hole function layer; 3. cathode; 4. light emitting layer; 5. electron transport layer; 6. substrate.

### DETAILED DESCRIPTION OF THE EMBODIMENTS

Exemplary embodiments will be described in detail herein, with the illustrations thereof represented in the drawings. When the following descriptions involve the drawings, like numerals in different drawings refer to like or similar elements unless otherwise indicated. The embodiments described in the following examples do not represent all embodiments consistent with the present disclosure. Rather, they are merely examples of apparatuses consistent with some aspects of the present disclosure as detailed in the appended claims.

The terms used in the present disclosure are for the purpose of describing particular embodiments only, and are not intended to limit the present disclosure. Unless otherwise defined, technical or scientific terms used in this disclosure should have ordinary meaning as understood by one of ordinary skill in the art to which the disclosure belongs. “First”, “second” and similar words used in the specification and claims of the present disclosure do not represent any order, quantity or importance, but are used only to distinguish different components. Likewise, similar words such as “one”, “a” or “an” do not represent a quantity limit, but represent that there is at least one. “Plurality”, “multiple” or “several” means two or more. Unless otherwise indicated, similar words such as “front”, “rear”, “lower” and/or “upper” are only for convenience of description, and are not limited to one position or one spatial orientation. Similar words such as “including” or “comprising” mean that an element or an item appearing before “including” or “comprising” covers elements or items and their equivalents listed after “including” or “comprising”, without excluding other elements or items. Similar words such as “connect” or “connected with each other” are not limited to physical or mechanical connections, and may include electrical connections, whether direct or indirect. Terms determined by “a/an”, “the” and “said” in their singular forms in the present disclosure and the appended claims are also intended to include plural forms unless clearly indicated otherwise in the context. It should also be understood that the term “and/or” as used herein refers to and includes any or all possible combinations of one or more associated listed items.

Embodiments of the present disclosure provide an organic compound for preparing a hole transport layer or a hole injection layer. A structure of the organic compound is as shown in Formula 1, Formula 2 or Formula 3:

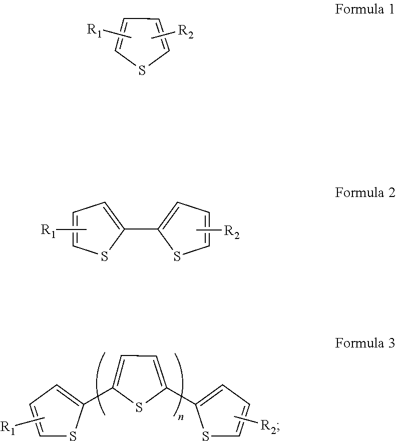

where n is an integer greater than or equal to 1; R₁ and R₂ are each independently selected from hydrogen, a substituted or unsubstituted carbazolyl group, and a substituted or unsubstituted diphenylamine group; R₁ and R₂ are not simultaneously hydrogen; and R₁ and R₂ cannot be bonded to one carbon atom.

The organic compounds according to the embodiments of the present disclosure include one or more thienyl groups, and substituents R₁ and R₂ bonded to the thienyl groups. The thienyl groups can impart a hole transport property to materials. R₁ and R₂ are each independently selected from hydrogen, a substituted or unsubstituted carbazolyl group, and a substituted or unsubstituted diphenylamine group, and R₁ and R₂ are not simultaneously hydrogen, thereby imparting a hole injection property to the materials, further endowing the materials with both the hole transport and injection properties, and improving a hole transport rate. Application of the organic compound in preparing quantum dot light emitting devices can improve a carrier injection balance of the devices and increase performance of the devices.

The Formula 1 includes a monothienyl group, that is, one thienyl group, and the substituent R₁ and R₂ can be bonded to any carbon atom except a sulfur atom on the thienyl group. The Formula 2 includes a bis-thienyl group, that is, two thienyl groups, the substituent R₁ can be bonded to any carbon atom except a sulfur atom on one of the two thienyl groups, and the substituent R₂ can be bonded to any carbon atom except a sulfur atom on the other of the two thienyl groups. The Formula 3 includes a polythienyl group, that is, multiple thienyl groups, which are bonded in sequence, the substituent R₁ can be bonded to any carbon atom except a sulfur atom on a thienyl group located at a head end of the multiple thienyl groups, and the substituent R₂ can be bonded to any carbon atom except a sulfur atom on a thienyl group located at a tail end of the multiple thienyl groups. For example, a structure of the Formula 1 may be as shown in Formula 1-1, a structure of the Formula 2 may be as shown in Formula 2-1, and a structure of the Formula 3 may be as shown in Formula 3-1. The Formula 1-1, Formula 2-1 and Formula 3-1 are as follows:

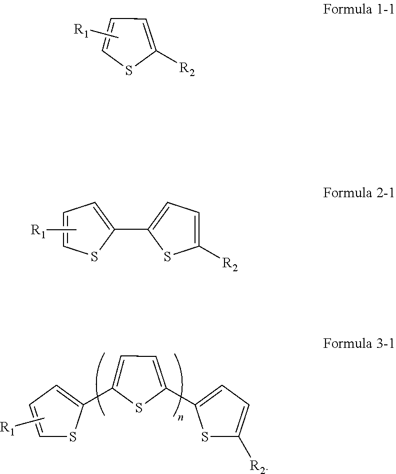

Compared with the Formula 1, in the Formula 1-1, R2 is bonded to a carbon atom adjacent to a sulfur atom on a thienyl group, and R₁ can be bonded to any one of the remaining three carbon atoms. Compared with the Formula 2, in the Formula 2-1, R₂ is bonded to a carbon atom adjacent to a sulfur atom on one of two thienyl groups, and R₁ can be bonded to any carbon atom on the other of the two thienyl groups. Compared with the Formula 3, in the formula 3-1, R₂ can be bonded to a carbon atom adjacent to a sulfur atom on a thienyl group located at a tail end, and R₁ is bonded to any carbon atom except a sulfur atom on a thienyl group located at a head end. Compared with the Formula 1, Formula 2 and Formula 3, synthesis difficulties of organic compounds of the Formula 1-1, Formula 2-1 and Formula 3-1 are reduced, thereby lowering preparation costs.

In some embodiments, a structure of the Formula 1-1 may be as shown in Formula 1-2, a structure of the Formula 2-1 may be as shown in Formula 2-2, and a structure of the Formula 3-1 may be as shown in Formula 3-2. The Formula 1-2, Formula 2-2 and Formula 3-2 are as follows:

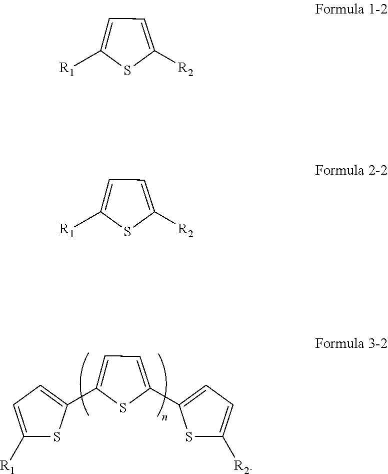

In the Formula 1-2, R₂ is bonded to one carbon atom adjacent to a sulfur atom on a thienyl group, and R₁ is bonded to the other carbon atom adjacent to the sulfur atom on the thienyl group. In the Formula 2-2, R₂ is bonded to a carbon atom adjacent to a sulfur atom on one of two thienyl groups, and R₁ is bonded to a carbon atom adjacent to a sulfur atom on the other of the two thienyl groups. In the formula 3-2, R₂ is bonded to a carbon atom adjacent to a sulfur atom on a thienyl group located at a tail end, and R₁ is bonded to a carbon atom adjacent to a sulfur atom on a thienyl group located at a head end. Compared with the Formula 1-1, Formula 2-1 and Formula 3-1, synthesis difficulties of organic compounds of the Formula 1-2, Formula 2-2 and Formula 3-2 are further reduced.

The substituents R₁ and R₂ are each independently selected from hydrogen or following groups:

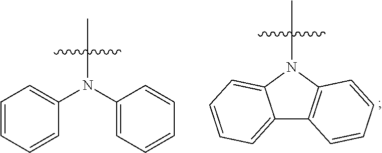

where

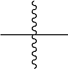

substituents R₁ and R₂ may be the same as or different from each other. In other embodiments of the present disclosure, R₁ or R₂ may have a substituent, and the substituent of R₁ or R₂ may be an alkyl group or an aryl group, but the present disclosure is not limited thereto.

N may be an integer less than or equal to 10, such as 1, 2, 3, 4, 5, 6, 7, 8, 9 or 10.

For example, the organic compound is selected from a group consisting of following structural formulas:

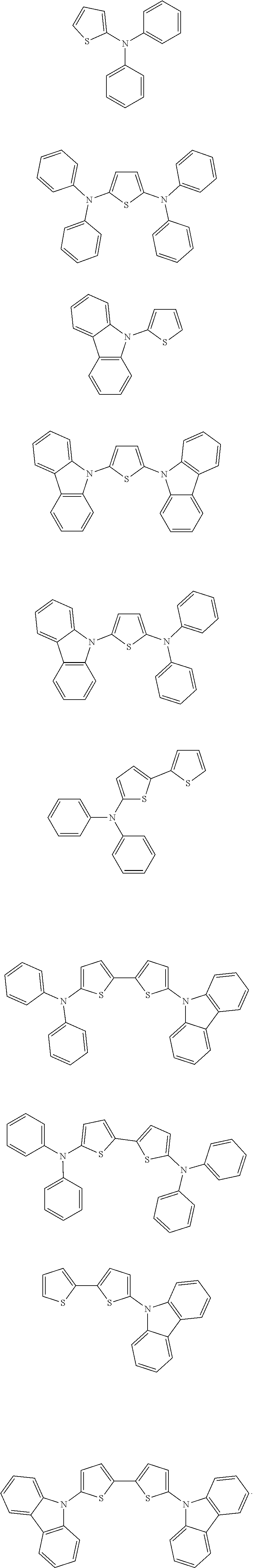

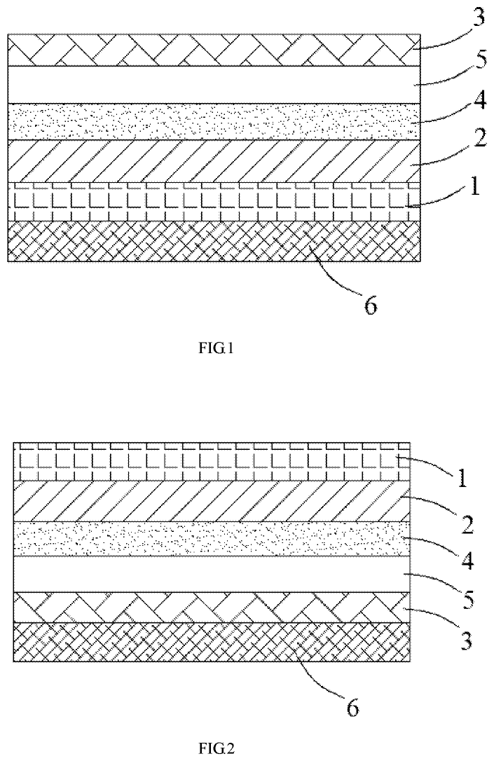

The embodiments of the present disclosure further provide a light emitting device. As shown in FIG. 1, the light emitting device may include an anode 1 and a cathode 3 disposed opposite to each other; a light emitting layer 4 disposed between the anode 1 and the cathode 3; and a hole function layer 2 disposed between the light emitting layer 4 and the anode 1. The hole function layer 2 may include the organic compound according to any one of the above embodiments.

Since organic compounds included in the light emitting device according to the embodiments of the present disclosure are the same as that in the above organic compound embodiments, they have the same beneficial effect, and the present disclosure will not repeat it here.

The hole function layer 2 may include a hole transport layer, and the hole transport layer may include an organic compound. The hole transport layer may be composed of the organic compound provided by the present disclosure, or other materials together with the organic compound provided by the present disclosure. The hole function layer 2 may include a hole injection layer, and the hole injection layer may include an organic compound. The hole injection layer may be composed of the organic compound provided by the present disclosure, or other materials together with the organic compound provided by the present disclosure. Since the organic compounds of the present disclosure have both the hole transport and injection properties, the hole function layer 2 may include only one of the hole injection layer and the hole transport layer, which simplifies a structure of the light emitting device, reduces technological difficulties, and saves its cost. In addition, the simplified structure of the light emitting device is more suitable for a print patterned process, while a carrier transport distance can be shortened, thereby reducing a resistance of the light emitting device, lowering a turn-on voltage of the light emitting device, and helping to improve a service life of the light emitting device.

The light emitting device may be an organic electroluminescent device, that is, the light emitting layer 4 is an organic electroluminescent layer. And in other embodiments, the light emitting device may alternatively be a quantum dot light emitting device, that is, the light emitting layer 4 is a quantum dot (QD) layer. As shown in FIG. 1, taking the light emitting device being a quantum dot light emitting device as an example, the quantum dot light emitting device has an upright structure, and includes substrate 6, anode 1, hole function layer 2, light emitting layer 4, electron transport layer 5 and cathode 3 which are arranged in a stacked manner. As shown in FIG. 2, the quantum dot light emitting device may have an inverted structure, and includes substrate 6, cathode 3, electron transport layer 5, light emitting layer 4, hole function layer 2 and anode 1 which are arranged in a stacked manner. In quantum dot light emitting devices with the upright structure and/or inverted structure, the hole function layer 2 is in direct contact with the anode 1 and the light emitting layer 4, and the hole function layer 2 has a single-layer structure.

Materials for the electron transport layer 5 may include metal oxide semiconductor nanoparticles with high electron mobility, such as ZnO and ZnMgO. Since the hole function layer 2 in the quantum dot light emitting device has both the hole transport and injection properties, a hole transport rate is increased, and a carrier injection balance of the device is improved. An exciton recombination region is confined within the light emitting layer 4, which improves performance of the light emitting device.

The anode 1 may be made of the following anode materials, which are preferably materials having a large work function. Specific examples of the anode materials include: metals, such as nickel, platinum, vanadium, chromium, copper, zinc and gold, or alloys thereof; metal oxides, such as zinc oxide, indium oxide, indium tin oxide (ITO) and indium zinc oxide (IZO); combinations of metal and metal oxide, such as ZnO: Al or SnO₂: Sb; or conductive polymers, such as poly (3-methylthiophene), poly[3,4-(ethylidene-1,2-dioxy)thiophene] (PEDT), polypyrrole and polyaniline, but they are not limited thereto. In some embodiments, a metal electrode including indium tin oxide (ITO) is used as the anode 1.

The cathode 3 is made of the following cathode materials, which are materials having a small work function. Specific examples of the cathode materials include: metals, such as magnesium, calcium, sodium, potassium, titanium, indium, yttrium, lithium, gadolinium, aluminum, silver, tin and lead, or alloys thereof; or multilayer materials, such as LiF/Al, Liq/Al, LiO₂/Al, LiF/Ca, LiF/Al, and BaF₂/Ca, but they are not limited thereto. In some embodiments, a metal electrode including an Mg-Ag alloy is used as the cathode 3.

Taking the light emitting layer 4 being a quantum dot layer as an example, the quantum dot layer may include a quantum dot structure. The quantum dot structure includes a core, and a shell covering the core. Optionally, the core is made of materials selected from a group consisting of CdS, CdSe, ZnSe, InP, CuInS, (Zn)CulnS, (Mn)CuInS, AgInS, (Zn)AgInS, CulnSe, CuInSeS, PbS, organic-inorganic perovskite materials, inorganic perovskite materials and any combination or alloy thereof. In some embodiments, the shell is made of materials selected from a group consisting of ZnS, ZnSeS, CdS, organic-inorganic perovskite materials, inorganic perovskite materials and any combination or alloy thereof

### Synthesis of Compounds

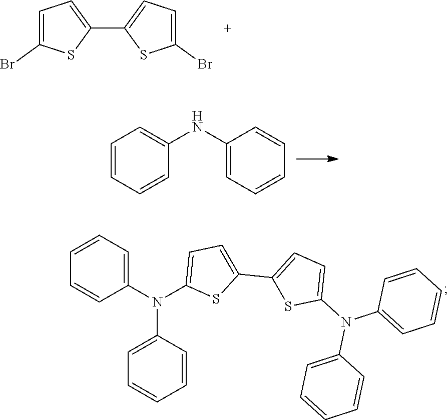

5,5′-dibromo-2,2′-bisthiophene (3.24g, 0.01mol), diphenylamine (3.38g, 0.02mo1), sodium tert-butoxide (1.92 g, 0.02 mol), tri-tert-butylphosphine (0.2 g, 0.001 mo1) and palladium acetate (0.23 g, 0.001 mol) were weighed and dissolved in a toluene (25 mL) solvent to form a mixed solution. The formed mixed solution was heated to 120° C. with magnetic stirring and refluxed for 12 h. After the reaction was finished, the mixed solution was cooled to room temperature. The mixed solution cooled to the room temperature was quickly poured into refrigerated methanol (150 mL) and stirred to produce a precipitate. The precipitate was collected by filtration. The precipitate was dissolved with a small amount of toluene, and the product was purified by column chromatography to obtain compound A. An eluent used in the column chromatography was petroleum ether: toluene (5:2). Hydrogen spectrum/1H-NMR data of the product purified by the column chromatography was as follows:

1H NMR (CDC1₃): δ=6.41 (d, 2H), 6.65 (d, 2H), 6.81 (t, 4H), 7.08 (t, 8H), 7.16 (d, 8H).

The above synthesis reaction is carried out under the protection of argon, and the solvent used is an ultra-dry anhydrous and anoxic solvent, for example, toluene, tetrahydrofuran and diethyl ether are refluxed and co-evaporated with a sodium and benzophenone system. The above synthesis method is only an example of the present application, but does not limit the present application.

Taking the light emitting device being a quantum dot light emitting device as an example, a method for preparing the quantum dot light emitting device is as follows:

### I. Solution Method

The organic compound according to the present disclosure was dissolved in a toluene solvent to prepare an organic compound solution with a concentration of 0.1%-10%.

Taking a red light quantum dot light emitting device as an example, as shown in FIG. 1, the specific process of the solution method is as follows:

1. first use anhydrous ethanol to ultrasonically clean an ITO glass substrate for 15 minutes, then use deionized water to ultrasonically clean the ITO glass substrate for 15 minutes and dry the substrate, next, use ultraviolet light to irradiate the ITO glass substrate for 10 minutes, so as to improve a surface work function of the ITO glass substrate, where the ITO glass substrate is used as the anode 1 of the light emitting device;

2. spin-coat the organic compound solution on a surface of the anode 1, heat and anneal at 120° C. for 15 minutes to form a uniform thin film on the surface of the cathode 3, where the thin film can be used as the hole function layer 2;

3. spin-coat a solution having a quantum dot structure on a surface of the hole function layer 2, heat and anneal at 100° C. for 15 minutes to form a uniform light emitting layer 4 on the surface of the hole function layer 2;

4. spin-coat a solution including ZnO nanoparticles on a surface of the light emitting layer 4, heat and anneal at 100° C. for 15 minutes to form a uniform ZnO thin film on the surface of the light emitting layer 4, that is, the electron transport layer 5;

5. form an Al electrode, that is, the cathode 3, on a surface of the electron transport layer 5 by vacuum evaporation.

### II. Evaporation Method

Taking a red light quantum dot light emitting device as an example, as shown in FIG. 2, the specific process of the evaporation method is as follows:

1. first use anhydrous ethanol to ultrasonically clean an ITO glass substrate for 15 minutes, then use deionized water to ultrasonically clean the ITO glass substrate for 15 minutes and dry the substrate, next, use ultraviolet light to irradiate the ITO glass substrate for 10 minutes, so as to improve a surface work function of the ITO glass substrate, where the ITO glass substrate is used as the cathode 3 of the light emitting device;

2. spin-coat a solution including ZnO nanoparticles on a surface of the cathode 3, heat and anneal at 100° C. for 15 minutes to form a uniform ZnO thin film on the surface of the cathode 3, that is, the electron transport layer 5;

3. spin-coat a solution having a quantum dot structure on a surface of the electron transport layer 5, heat and anneal at 100° C. for 15 minutes to form a uniform light emitting layer 4 on a surface of the ZnO thin film;

4. deposit organic compound materials according to the present disclosure on a surface of the light emitting layer 4 by vacuum evaporation to form the hole function layer 2;

5. form an Al electrode, that is, the anode 1, on a surface of the hole function layer 2 by vacuum evaporation.

The above are only preferred embodiments of the present disclosure, which are not intended to make any formal limitation on the disclosure. Although the present disclosure has been disclosed as above in the preferred embodiments, these preferred embodiments are not intended to limit the present disclosure, and any person skilled in the art, without departing from the scope of the technical solutions of the present disclosure, can make some changes or modifications to the technical contents disclosed above as equivalent embodiments with equivalent changes. However, without departing from the contents of the technical solutions of the present disclosure, any simple revisions, equivalent changes and modifications made to the above embodiments based on the technical essence of the present disclosure still fall within the scope of the technical solutions of the present disclosure.

1. An organic compound, wherein a structure of the organic compound is as shown in Formula 1, Formula 2 or Formula 3: 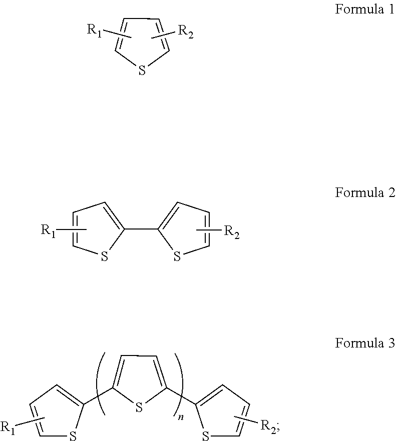
wherein n is an integer greater than or equal to 1;
each of R₁ and R₂, independently, is selected from hydrogen, a substituted or unsubstituted carbazolyl group, and a substituted or unsubstituted diphenylamino group, and R₁ and R₂ are not simultaneously hydrogen.

2. The organic compound according to claim 1, wherein the structure of the organic compound is as shown in Formula 1-1, Formula 2-1 or Formula 3-1:

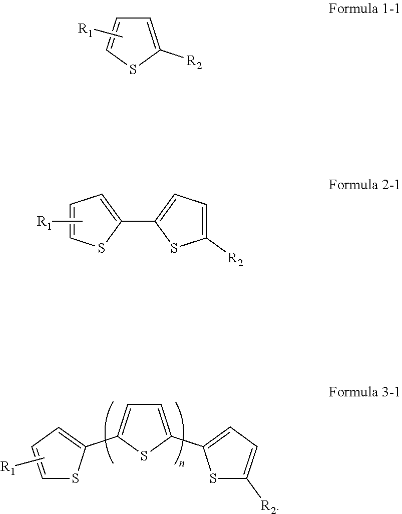

3. The organic compound according to claim 2, wherein the structure of the organic compound is as shown in Formula 1-2, Formula 2-2 or Formula 3-2:

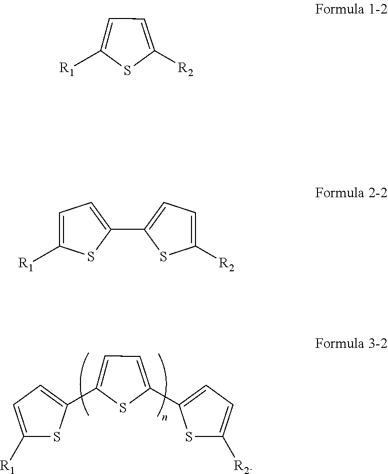

4. The organic compound according to claim 1, wherein each of R₁ and R₂, independently, is selected from hydrogen or following groups: 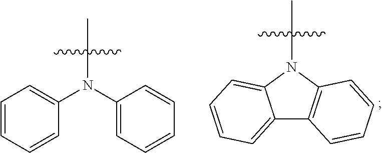 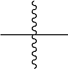
wherein represents a chemical bond.

5. The organic compound according to claim 1, wherein n is an integer less than or equal to 10.

6. The organic compound according to claim 1, wherein the organic compound is selected from a group consisting of following structural formulas:

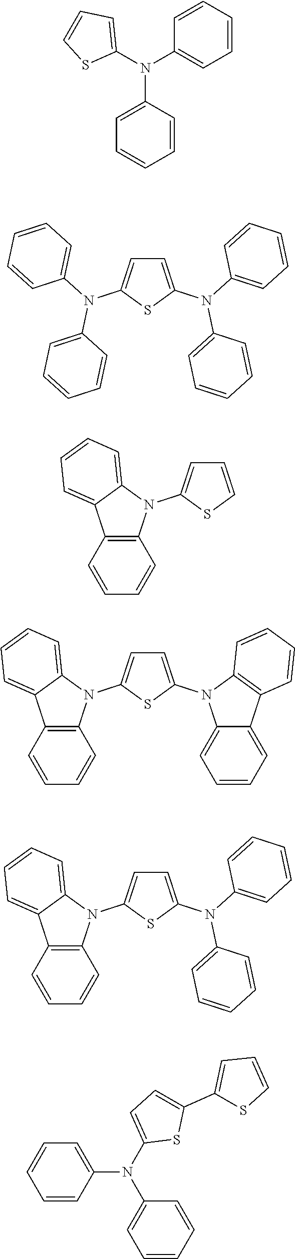

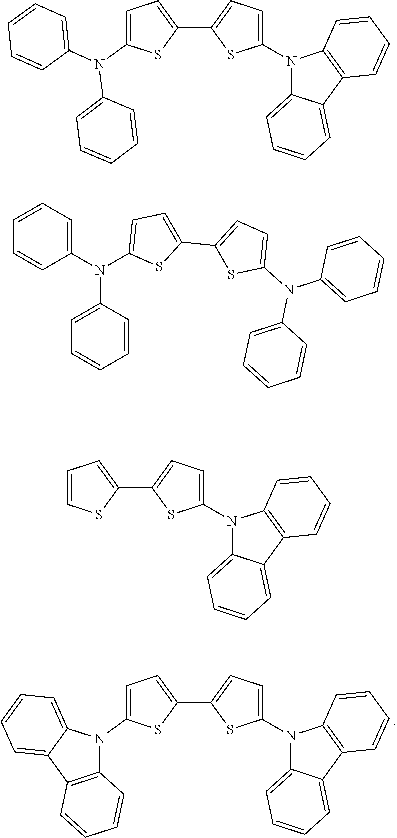 [SMILES: c1ccc(N(c2ccccc2)c2ccc(-c3ccc(-n4c5ccccc5c5ccccc54)s3)s2)cc1 ; c1ccc(N(c2ccccc2)c2ccc(-c3ccc(N(c4ccccc4)c4ccccc4)s3)s2)cc1 ; c1csc(-c2ccc(-n3c4ccccc4c4ccccc43)s2)c1 ; c1ccc2c(c1)c1ccccc1n2-c1ccc(-c2ccc(-n3c4ccccc4c4ccccc43)s2)s1]

7. The organic compound according to claim 1, wherein R₁ and R₂ are the same as each other.

8. A light emitting device, comprising:
an anode and a cathode disposed opposite to each other;
a light emitting layer disposed between the anode and the cathode;
a hole function layer disposed between the anode and the light emitting layer and including an organic compound, wherein a structure of the organic compound is as shown in Formula 1, Formula 2 or Formula 3: 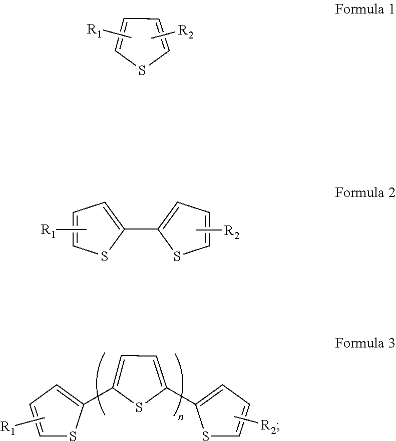
wherein n is an integer greater than or equal to 1;
each of R₁ and R₂, independently, is selected from hydrogen, a substituted or unsubstituted carbazolyl group, and a substituted or unsubstituted diphenylamino group, and R₁ and R₂ are not simultaneously hydrogen.

9. The light emitting device according to claim 8, wherein the hole function layer comprises a hole transport layer, and the hole transport layer comprises the organic compound.

10. The light emitting device according to claim 8, wherein the hole function layer comprises a hole injection layer, and the hole injection layer comprises the organic compound.

11. The light emitting device according to claim 8, wherein the light emitting device is a quantum dot light emitting device.

12. The organic compound according to claim 2, wherein each of R₁ and R₂, independently, is selected from hydrogen or following groups: 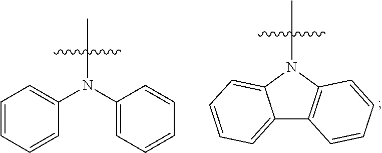
where 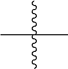
represents a chemical bond.

13. The organic compound according to claim 3, wherein each of R₁ and R₂, independently, is selected from hydrogen or following groups: 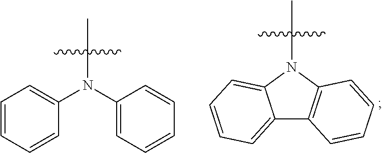
where 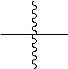
represents a chemical bond.

14. The organic compound according to claim 2, wherein n is an integer less than or equal to 10.

15. The organic compound according to claim 3, wherein n is an integer less than or equal to 10.

16. The organic compound according to claim 2, wherein the organic compound is selected from a group consisting of following structural formulas:

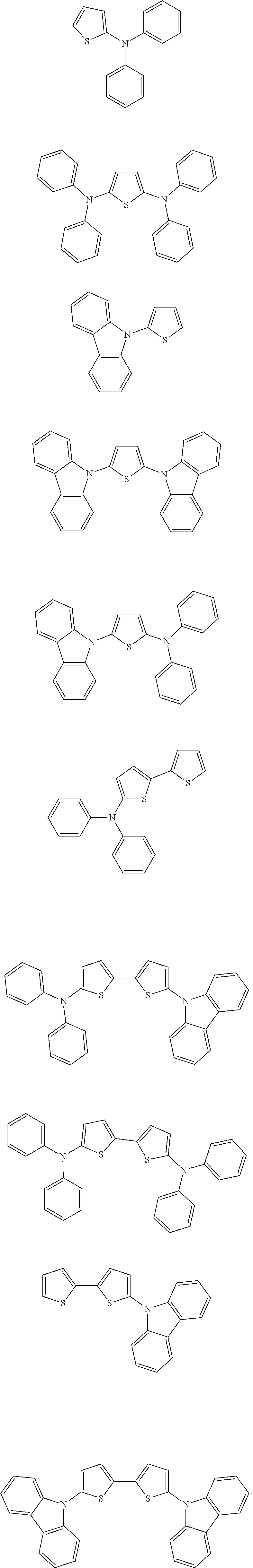

17. The organic compound according to claim 3, wherein the organic compound is selected from a group consisting of following structural formulas:

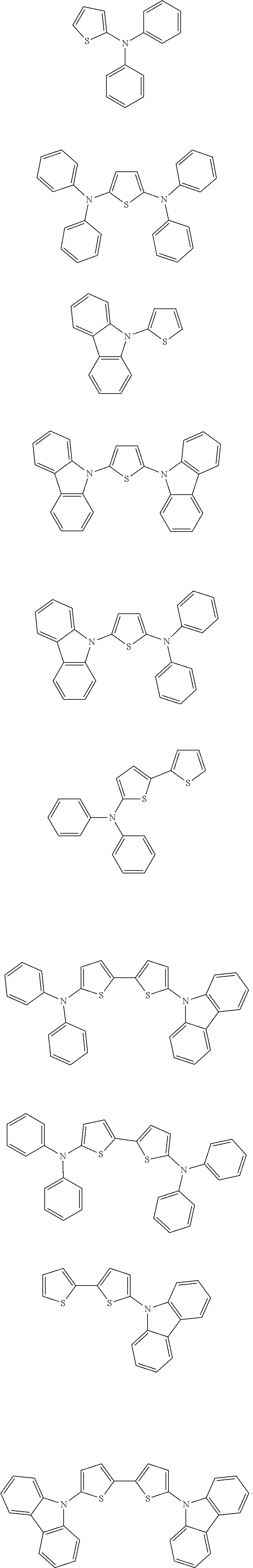

18. The organic compound according to claim 2, wherein R₁ and R₂ are the same as each other.

19. The organic compound according to claim 3, wherein R₁ and R₂ are the same as each other.

20. A light emitting device, comprising:
an anode and a cathode disposed opposite to each other;
a light emitting layer disposed between the anode and the cathode;
a hole function layer disposed between the anode and the light emitting layer and including an organic compound, wherein a structure of the organic compound is as shown in Formula 1-1, Formula 2-1 or Formula 3-1: [chemical structure]
